(19) United States
(12) Patent Application Publication
(10) Pub. No.: US 2019/0175419 A1
(43) Pub. Date: Jun. 13, 2019

(54) Dissolving Hygienic Male Urine Wipe

(71) Applicant: Greg Alan Reitman, Boise, ID (US)

(72) Inventor: Greg Alan Reitman, Boise, ID (US)

(21) Appl. No.: 15/837,680

(22) Filed: Dec. 11, 2017

(51) Int. Cl. A61F 13/15; A61K 8/02

(52) U.S. Cl. CPC A61F 13/15211; A47K 2010/3266; A61K 8/0208

(57) ABSTRACT

An aqueous soluble wipe designed to remove excess micturition from the end of a male urethra. The wipe, due to its absorption abilities reduces post urination leakage. Due to said wipes solubility characteristics, used wipe may be readily disposed of in most common urinal settings without constraints to flush ability in modern urinal settings.

### CROSS REFERENCE TO RELATED APPLICATIONS

This application claims priority to and the benefit of the filing date of U.S. Provisional Application No. 62/433,353, filed on Dec. 13, 2016, entitled “Dissolving Hygienic Wipes”, which is incorporated by reference in its entirety.

### FIELD OF THE INVENTION

The present patent specification relates to a water soluble, bio-degradable, aqueous soluble wipe that a male can use for removal of excess urine from his urethra after urination. More particularly, the wipe will reduce post urinary drip and micturition and can be disposed of in plumbed urinals and dissolves completely when diluted with a quantity of aqueous solution as to not impair plumbed drains or cisterns associated with urinals.

### BACKGROUND

In common household lavatory settings, there is commonly displayed in near proximity to the commode, tissue paper, typically in perforated “roll” form. In such settings, males can utilize such tissue to remove excess urine after urination to provide a more hygienic feeling through dispersing excess urine from the urethra by utilizing a “pressing” or “dabbing” action; or a combination thereof, of excess urine into said material and disposing of used material into a functionally plumbed toilet.

In situations where urination occurs in a plumbed urinal setting, typically found in public restrooms, materials such as hygienic “wipes” or common toilet paper are not commonly, readily available. This lack of hygienic opportunity creates an environment where excess micturition is possibly dispersed onto the floor or plumbing fixtures creating unsanitary conditions and possibly unpleasant odor. Further, having the lack of ability to provide a disposal method for excess urine may create possible staining or spotting of clothing.

The lack of hygienic wipes near a urinal setting is predominantly due to the common use of urinal “screens” that prevent non-soluble objects such as gum, cigarette butts, candy, chewing tobacco pouches or any other non-soluble item to be introduced into the drain portion of a urinal. These devices can commonly contain deodorizers to reduce the smell of urine and are commonly known as “urinal cakes”. These screens or cakes readily trap any non-soluble item and, given enough items, block the drainage of the urinal bowl.

Recently, the development of urine wipes designed for males has introduced wipes that are bio-degradable. The drawback to degradability is the presence of time needed for complete solubility. Most wipes presented in the marketplace do not dissolve in a rapid enough fashion to allow for flush ability in a urinal setting.

Also, it is commonly recognized that any substrate introduced into a urinal that is not rapidly soluble will increase the opportunity for urinal overflow due to the lower bowl height existing in front of modern manufactured urinals.

Further, it is recognized that with modern conservancy initiatives within the plumbing industry, there is more and more emphasis on manufacturing urinals that are eliminating waste with a reduced flush capacity (<1 gallon per flush).

Utilization of waterless commercial urinals is increasing, thus further reducing the likelihood of owner/operators of urinals to implement any type of hygienic wipe near them for fear of introduction into the plumbing system and thus creating a blockage.

Therefore, the need exists in the field for a hygienic wipe that assists males with the removal of excess urine from a male urethra and that can be disposed of in the urinal without the risk to owner/operator of said urinal of creating blockage in the drain or cistern system.

This need is further exemplified in the reduced GPM rates of modern plumbed urinals and/or the presence of waterless urinals.

### BRIEF SUMMARY OF THE INVENTION

In preferred embodiments, the disposable wipe will be between 2-3″ in width and 3-4″ in length. The wipe would be dispensed from a gravity feed or upright dispenser in standard V-fold fashion. Each sheet would be interwoven with the next, in order to provide a hygienic, single wipe in an ongoing supply method.

The preferred embodiment of said wipe would be composed from a combination of sodium carboxymethyl cellulose and wood pulp. The combination of ingredients would vary based on the thickness, opaqueness, whiteness and absorbency required.

Each sheet would have the capacity to hold 0.1 m/l to 1 m/l of fluid before reaching the saturation point and subsequent point of dilution.

The user could utilize multiple wipes in removal of urine and with the multiplicity of wipes, gain incrementally more absorption ability and rigidity of the wipe(s) prior to the wipe(s) reaching a point of dilution.

Males would use the disposable urine wipe to “dab” or “press” excess urine into the wipe and dispose of the material directly into the urinal bowl.

### DETAILED DESCRIPTION OF THE INVENTION

In describing the invention, it will be understood that the terminology used is for the purpose of describing embodiments, components and/or skills required to create the art belonging to the invention.

The terminology used herein is for the purpose of describing the art belonging to the invention and is not intended to be limiting to the invention.

The present disclosure is to be considered as an exemplification of the invention, and is not intended to limit the invention to the specific embodiments illustrated by the figure(s) or description below.

The present invention provides an absorbent wipe for males to remove post micturition urine from the end of the urethra.

In preferred embodiments of said wipe, composition would be derived from a combination of sodium carboxy methyl cellulose (CAS # 9004-32-4) and wood pulp (CAS # 65996-61-4). The combination would vary based on thickness, opaqueness, whiteness and absorbency required. In preferred embodiments, said wipe would by one ply in nature.

Each wipe would have the absorption capacity to hold 0.1 m/l to 1 m/l of fluid before reaching the saturation point and subsequent point of dilution.

Each sheet would have a surface area 6″ sq. to 12″ sq. and preferably be rectangular in shape though other shapes may be utilized.

Users can utilize multiple wipes and with the multiplicy of the device, gain incrementally more absorption ability prior to the wipe reaching a point of dilution.

FIG. (1) represents the wipes in interwoven, V-fold fashion. The presented fashion would allow the wipes to be dispensed from a gravity fed or upright display located near the urinal. Each sheet would be interwoven with the next in order to provide a hygienic, single wipe in an ongoing supply fashion.

I claim an embodiment (a wipe) configured for removing excess urine from a male urethra after urination comprising:

1. a combination of sodium carboxymethyl cellulose and wood pulp that is soluble and dispersible within a public urinal environment.

2. The wipe of claim 1, being single ply in nature

3. The wipe of claim 1, having a length between 3-4″ and width between 2-3″.

4. The wipe of claim 1, having a thickness between 0.03-0.05 mm.

5. The wipe of claim 1, having an absorption capacity between 0.1 m/l and 1 ml.
